(19) United States
(12) Patent Application Publication
(10) Pub. No.: US 2015/0150497 A1
(43) Pub. Date: Jun. 4, 2015

(54) INTRAUTERINE DEVICE

(71) Applicant: Mor Research Applications Ltd., Tel-Aviv (IL)

(72) Inventor: Chen Goldchmit, Nes Ziona (IL)

(21) Appl. No.: 14/415,591

(22) PCT Filed: Jul. 18, 2013
(86) PCT No.: PCT/IL2013/050608

§ 371 Date: Jan. 18, 2015

Related U.S. Application Data
(60) Provisional application No. 61/672,778, filed on Jul. 18, 2012.

(51) Int. Cl. A61B 5/00; A61B 8/12; A61B 1/303; A61B 1/012; A61B 1/015; A61B 1/06; A61B 1/018; A61B 17/32; A61B 1/00; A61B 8/08; A61B 8/00

(52) U.S. Cl. CPC A61B 5/4325; A61B 8/0858; A61B 8/12; A61B 1/303; A61B 1/0125; A61B 8/445; A61B 1/063; A61B 1/018; A61B 8/4494; A61B 8/463; A61B 8/5207; A61B 17/320016; A61B 1/0005; A61B 1/015; A61B 2017/0034

(57) ABSTRACT

According to an aspect of some embodiments of the present invention there is provided a probe sized for insertion into a uterus, the device comprising at least one ultrasound element adapted to emit ultrasonic energy, the ultrasound element disposed on a distal end of the device; at least one ultrasound receiver adapted to receive ultrasound energy, the ultrasound receiver disposed on a distal end of the device; and circuitry adapted to automatically estimate a thickness of a uterine wall according to the received ultrasound energy.

According to an aspect of some embodiments of the present invention there is provided a method of treating an inner wall of a uterus comprising automatically measuring a thickness of a uterine wall according to ultrasound energy emitted from inside the uterus; and providing an output of the thickness.

### RELATED APPLICATIONS

This application claims the benefit, also under 35 USC 119(e), of U.S. provisional application Ser. No. 61/672,778 filed 18 Jul. 2012, the disclosure of which is incorporated herein by reference.

### FIELD AND BACKGROUND OF THE INVENTION

The present invention, in some embodiments thereof, relates to an intrabody probe and, more particularly, but not exclusively, to an intrauterine ultrasound probe.

Kazem Nouri et al. “Reproductive outcome after hysteroscopic septoplasty in patients with septate uterus—a retrospective cohort study and systematic review of the literature”, Reprod Biol Endocrinal. 2010; 8:52. Published online 2010 May 21. Disclose “Sixty-four women underwent hysteroscopic septoplasty. In 2/64 (3%) women, intraoperative uterine perforation occurred. Including our own data, we identified 18 studies investigating the effect of septoplasty on reproductive outcome in 1501 women. The overall rate of intra- and postoperative complications was 1.7% (23/1324) and the overall rate of re-hysteroscopy was 6% (79/1324).”

CN101518455(A) entitled Hard ultrasonic hysteroscope system discloses “The hard ultrasonic hysteroscope system comprises a hard hysteroscope, a hysteroscope sheath tube, a micro ultrasound probe and a hysteroscope camera system, wherein the hysteroscope sheath tube is connected with the hard hysteroscope the micro ultrasound probe can provide real-time ultrasonic scanning”.

CN201752417U entitled Integrated hard ultrasonic hysteroscope system discloses “The hard hysteroscope comprises a hysteroscope body and a hysteroscope sheath catheter connected with the hysteroscope body, a miniature ultrasonic probe, an optical lens, light guide optical fibers and an operating passage outlet are disposed at the end of the hard hysteroscope.”

CN101828931(A) entitled Miniature ultrasonic electronic hysteroscope system discloses “a micro ultrasonic probe is also inserted in an appliance channel of the endoscope main body of the hard electronic hysteroscope and the micro ultrasonic probe is connected with a micro ultrasonic system processing host and the monitor.”

Additional background art includes: WO2008046031, WO2009018351, WO2005086737.

### SUMMARY OF THE INVENTION

An aspect of some embodiments of the invention relates to an ultrasound probe adapted to automatically estimate the thickness of a tissue. In an exemplary embodiment, the uterine wall is measured from inside the uterus. Optionally, the thickness measurement includes pathologies (e.g., septum, fibroids).

According to an aspect of some embodiments of the present invention there is provided a probe sized for insertion into a uterus, the device comprising:

at least one ultrasound element adapted to emit ultrasonic energy, the ultrasound element disposed on a distal end of the device;

at least one ultrasound receiver adapted to receive ultrasound energy, the ultrasound receiver disposed on a distal end of the device; and

circuitry adapted to automatically estimate a thickness of a uterine wall according to the received ultrasound energy.

According to some embodiments of the invention, the circuitry is non-imaging.

According to some embodiments of the invention, the circuitry is further configured to only measure the thickness.

According to some embodiments of the invention, the probe is sized for insertion into a lumen of a hysteroscope.

According to some embodiments of the invention, the uterine wall thickness comprises a thickness of a uterine septum and a myometrium.

According to some embodiments of the invention, the at least one ultrasound element is arranged to be forward facing so that the ultrasound energy is directed to intersect a surgical site on the uterine wall.

According to some embodiments of the invention, the ultrasound element is arranged so that the ultrasound energy is directed within a visual field on the uterine wall.

According to some embodiments of the invention, the ultrasound element is arranged to image an area no larger than about 100 mm².

According to some embodiments of the invention, the ultrasound element is arranged to produce an ultrasound beam of frequency from about 3-12 Mhz.

According to some embodiments of the invention, the probe is coupled to at least one of a visual element and a cutting element so that the probe moves together with the visual element and the cutting element to maintain an intersection of an ultrasound sensing area and at least one of a visual field and a surgical field, during movement of the probe.

According to some embodiments of the invention, the probe of claim 1 further comprises an output element in electrical communication with the circuitry, the output element adapted to provide at least one of visual and auditory output of the thickness.

According to some embodiments of the invention, the probe of claim 1 further comprises a colored light source positioned coaxially with the at least one ultrasound receiver so that a colored light field at least partially overlaps with a region of the wall being measured.

According to some embodiments of the invention, the probe is sized for insertion through an undilated cervix.

According to some embodiments of the invention, a device for insertion into a uterus comprises: a probe; at least one visual element adapted to provide visual images of a portion of a uterine wall, the visual element positioned so that a visual field encompass an ultrasound imaging field of the uterine wall; and at least one lumen sized for insertion of a surgical tool. Optionally, the at least one lumen is positioned so that the surgical tool treats the uterine wall within the visual field and within the ultrasound imaging field. Optionally or additionally, the device is sized for insertion into the uterus through a cervix. Optionally or additionally, the device is flexible for improved maneuverability within the uterus.

According to an aspect of some embodiments of the present invention there is provided a method of treating an inner wall of a uterus comprising:
automatically measuring a thickness of a uterine wall according to ultrasound energy emitted from inside the uterus; and
providing an output of the thickness.

According to some embodiments of the invention, automatically measuring is performed without producing an image.

According to some embodiments of the invention, automatically measuring further comprises detecting a near wall of the uterus according to ultrasound energy emitted from inside the uterus, the ultrasound energy emitted from an emitter not in contact with the wall; detecting a far wall of the uterus according to the emitted ultrasound energy; and estimating the thickness according to a distance between the near and far walls.

According to some embodiments of the invention, the method further comprises removing tissue from the inner wall of the uterus according to the output.

According to some embodiments of the invention, automatically measuring is performed during the removing tissue.

According to some embodiments of the invention, the removing tissue and the providing the output are repeated in an alternating manner or simultaneously.

According to some embodiments of the invention, the method further comprises providing visual output of an area of removed tissue and the measured uterine wall.

According to some embodiments of the invention, providing the output comprises providing an absolute measurement of the thickness.

According to some embodiments of the invention, providing the output comprises providing output according to the thickness being above or below a safety threshold.

According to some embodiments of the invention, providing the output comprises providing output according to the thickness being above or below a baseline.

According to some embodiments of the invention, the method further comprises distending the uterus by inserting a fluid into the uterus.

According to some embodiments of the invention, automatically estimating comprises estimating without contacting the uterus wall with an ultrasound emitter.

According to some embodiments of the invention, measuring comprises measuring the thickness with an accuracy of +/−3 mm.

According to some embodiments of the invention, measuring comprises measuring a plurality of regions.

According to an aspect of some embodiments of the present invention there is provided a method of aligning a device to measure thickness of a uterine wall, the method comprising aligning a visual field of the uterine wall with an ultrasound field so that tissue one or more regions of the uterine wall being measured lie within the visual field.

According to some embodiments of the invention, the method further comprises aligning a cutting tool with the measured regions so that tissue being cut is also being measured.

According to some embodiments of the invention, the method further comprises monitoring the alignment during a tissue removal procedure.

According to some embodiments of the invention, monitoring comprises detecting sudden changes in thickness.

According to some embodiments of the invention, the method further comprises re-aligning the visual field and the ultrasound field to maintain the alignment.

According to some embodiments of the invention, aligning comprises adjusting spread of an ultrasound field.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Implementation of the method and/or system of embodiments of the invention can involve performing or completing selected tasks manually, automatically, or a combination thereof. Moreover, according to actual instrumentation and equipment of embodiments of the method and/or system of the invention, several selected tasks could be implemented by hardware, by software or by firmware or by a combination thereof using an operating system.

For example, hardware for performing selected tasks according to embodiments of the invention could be implemented as a chip or a circuit. As software, selected tasks according to embodiments of the invention could be implemented as a plurality of software instructions being executed by a computer using any suitable operating system. In an exemplary embodiment of the invention, one or more tasks according to exemplary embodiments of method and/or system as described herein are performed by a data processor, such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-1D are simplified schematics of anatomical variations of the uterus, useful to help understand an exemplary embodiment of the invention;

FIG. 2 is a simplified schematic showing the probe inside a uterus, in accordance with an exemplary embodiment of the invention;

FIG. 3 is a flowchart of a method of measuring the uterine wall, in accordance with an exemplary embodiment of the invention;

FIG. 4 is a flowchart of a method of treating a patient, in accordance with an exemplary embodiment of the invention;

FIGS. 5A-5D are some schematics of the distal tip of a sheath for insertion in the uterus, in accordance with an exemplary embodiment of the invention;

FIG. 6 is a schematic of the visual field inside the uterus, in accordance with an exemplary embodiment of the invention; and

FIG. 7 is a flowchart of a method of alignment of the system, in accordance with an exemplary embodiment of the invention.

### DESCRIPTION OF SPECIFIC EMBODIMENTS OF THE INVENTION

The present invention, in some embodiments thereof, relates to an intrabody probe and, more particularly, but not exclusively, to an intrauterine ultrasound probe.

An aspect of some embodiments of the invention relates to an intrabody probe adapted to estimate the thickness of a tissue. In an exemplary embodiment of the invention, the uterine wall is measured from inside the uterus (e.g., cervical access). Optionally, the measurement is performed using ultrasound energy. Optionally, the measurement is performed automatically, for example, by a controller.

In an exemplary embodiment of the invention, the probe is designed for use during operative procedures. Alternatively or additionally, the probe is used for diagnostic purposes.

In an exemplary embodiment of the invention, the probe is adapted to only measure the tissue thickness.

In an exemplary embodiment of the invention, the probe is non-imaging, for example, the transmitted and/or received ultrasound energy is insufficient to produce and/or display an image. Optionally, the thickness is displayed visually (e.g., a number on a screen) and/or audibly (e.g., beeps, a voice says the thickness).

In an exemplary embodiment of the invention, the thickness of the uterine wall comprises the myometrium. Optionally or additionally, the thickness of the uterine wall comprises abnormal tissues (e.g., congenital defects, tumors, trauma outcomes), for example; a uterine septum (e.g., regardless of the histological makeup of the septum), a fibroid (e.g., submucous), uterine adhesions (e.g., a result of traumatic operative hysteroscopy). Alternatively, the thickness of the wall is measured during removal of normal tissues, for example, during operative hysteroscopy.

In an exemplary embodiment of the invention, the thickness of the uterine wall also includes the endometrium, for example, if significantly thick enough, for example, if thicker than about 1 mm, or about 2 mm, or about 3 mm, or about 5 mm, or other smaller, intermediate or larger values. Alternatively, the thickness of the endometrium is not measured and/or is not thick enough to be clinically relevant. In practice, the surgical procedure can be performed during the menstrual phase of the menstrual cycle when the endometrium is thinnest. Furthermore, in practice, thick endometrial lining is considered pathological and requires further investigation.

In an exemplary embodiment of the invention, the thickness of the uterine wall is measured within an accuracy tolerance of about +/−1 mm, or about +/−3 mm, or about +/−5 mm, or other smaller, intermediate or larger thicknesses. In practice, a high tolerance is not required to allow incision of the abnormal tissue (e.g., uterine septum) with a high degree of certainty that perforation of the uterine wall will not occur.

In some embodiments, the probe is adapted to fit inside a lumen of a sheath for insertion in the uterus (e.g., hysteroscope, resectoscope), for example, the probe is insertable in the sheath before the sheath is inserted in the uterus or once the sheath is properly positioned in the uterus. Alternatively, the probe is built into the hysteroscope. Alternatively, the probe is adapted for fitting to other intra-uterine devices, for example, inside a cannula used to perform operative hysteroscopy.

Alternatively, in some embodiments, the probe is adapted for insertion into the uterus on its own. Optionally, the probe is adapted for insertion through an undilated cervix, or partially dilated cervix. For example, the diameter of the probe is no more than about 1 mm or about 2 mm or about 3 mm, or about 4 mm, or about 5 mm, or about 7 mm, or other smaller, intermediate or larger sizes. Optionally, the probe inserted on its own into the uterus is adapted for providing good intra-uterine images (e.g., of the endometrium). Optionally, the images are of sufficient quality to diagnose, for example hyperplasia, cancer of the endometrium, endometrial polyps, residual trophoblastic tissue.

In some embodiments, the US probe is disposable.

In an exemplary embodiment of the invention, only the myometrium (and optionally the endometrium) is measured. Optionally, other tissues external to the uterus (e.g., uterine vessels, uterine ligaments, bladder, intestine, ovaries) are not measured. In some embodiments, distinct acoustic properties of the surrounding tissues are used to help differentiate between the uterine wall and the surrounding tissues, for example, air filled intestines, fluid filled bladder. Alternatively or additionally, abnormal tissues are also measured, for example, the septum, even if the abnormal tissues include a mix of tissue (e.g., muscle, connective and/or other tissues).

In an exemplary embodiment of the invention, the US element is not adapted to improve visual diagnosis. Optionally, the US element is not adapted to produce US images of the uterine wall that are of good enough quality for diagnosis, for example, of abnormalities of the wall. Optionally or additionally, the images produced by the US element are of sufficient quality to allow measurement of the uterine muscle thickness on the US image, for example, within the allowable tolerance.

In an exemplary embodiment of the invention, the ultrasound emitter is positioned so that the ultrasound energy intersects a visual field of view on the uterine wall. Optionally, optical imaging of the wall is provided by an optical element (e.g., fiber optic bundle). Alternatively or additionally, the US beam is parallel to the optical element field of view, but in close proximity to the visualized field, for example, within about 1 mm, about 2 mm, about 3 mm, or other smaller, intermediate or larger distances. Optionally, the measured wall thickness corresponds to the thickness of the tissue being viewed.

In an exemplary embodiment of the invention, the US emitter and the optical element are positioned and/or coupled so that changing the field of view relative to the uterine tissues (e.g., angular direction, lateral displacement) maintains measurements of the corresponding thickness of the visualized uterine wall.

In an exemplary embodiment of the invention, the ultrasound emitter is positioned so that the ultrasound energy is directed towards the surgical site (e.g., the portion of tissue being treated by a surgical tool). In an exemplary embodiment of the invention, the thickness of the uterine wall is measured at the surgical site, for example, if using scissors, the thickness of the wall is measured at the location of cutting.

In an exemplary embodiment of the invention, the ultrasound emitter and the surgical tool are positioned and/or coupled so that movement of the tool provides a corresponding movement of the ultrasound emitter. Optionally or additionally, the US emitter, the tool and the visual element are all coupled and/or positioned to move together and maintain the alignment of the visual, US and cutting axes within an allowed tolerance. In an exemplary embodiment of the invention, the cutting axis, the visual axis and the US axis are all aligned. Optionally, some misalignment is allowed.

In an exemplary embodiment of the invention, the ultrasound element is adapted to emit a forward facing beam of ultrasound energy. The beam does not diverge more than, for example, about 5 degrees (e.g., perpendicular from the emitter), or about 10 degrees, or about 15 degree, or about 30 degrees, or other smaller, intermediate or larger divergence angles. Optionally or additionally, the ultrasound element is adapted to emit a pencil thin beam of ultrasound energy. The tissue area being imaged is, for example, about 1 mm², or about 5 mm², or about 10 mm², or about 15 mm², about 20 mm², about 50, about 100 mm², about 200 mm², or other smaller, intermediate or larger values are used. In an exemplary embodiment of the invention, the area of uterine wall being sensed with US is not significantly larger than the surgical site. Alternatively, the area being measured is larger than the surgical site. In such a case, in some embodiments, several measurements of the thickness of the uterine wall are made within the US imaged area, with the thinnest value being the most significant value.

Optionally, the US beam is smaller than the visual field of view. Alternatively, most of the area being sensed with US overlaps with the visual field of view, for example, at least 50%, at least 70%, at least 85%, or other smaller, intermediate or larger values.

In an exemplary embodiment of the invention, the frequency of the emitted ultrasound energy is selected to penetrate at least the full thickness of the uterine wall, including the septum thickness. For example, at least 5 mm, at least 8 mm, at least 10 mm, at least 12 mm, at least 15 mm, at least 20 mm, or other smaller, intermediate or large thicknesses. The frequency selected is, for example, about 3-12 Mhz, or about 5-7.5 Mhz, or other smaller, intermediate or larger values.

In an exemplary embodiment of the invention, the probe is adapted to provide output related to the estimated thickness of the uterine wall, for example, visual output (e.g., colors, numbers) and/or audio output (e.g., beeps, verbal messages). Optionally, the output corresponds to the absolute measured value of the wall thickness. Alternatively or additionally, the output corresponds to the wall thickness being above or below a threshold, or in between one or more value ranges.

An aspect of some embodiments of the invention relates to a method of estimating the thickness of a tissue. In an exemplary embodiment of the invention, the thickness of the uterine wall (e.g., myometrium) is estimated. Optionally, one or more measurements of the wall thickness occur during resection of the uterine wall, for example, continuous monitoring or monitoring in bursts that alternate with cutting.

In an exemplary embodiment of the invention, the method comprises emitting US energy at the uterine wall, and measuring the returning ultrasound echoes. Optionally, the outer uterine wall is detected, for example, by a first returning echo. Optionally or additionally, the inner uterine wall is detected, for example, by a second returning echo. Optionally or additionally, the thickness of the wall is estimated by estimating the distance between the first and second echoes.

In an exemplary embodiment of the invention, the method comprises providing feedback (e.g., visual and/or audio) according to the estimated wall thickness. Optionally, the feedback comprises the thickness of the measured wall, for example, in millimeters. Alternatively or additionally, the feedback comprises providing permission to continue cutting (e.g., wall is thicker than a safe threshold value) or to stop cutting (e.g., wall is thinner than the safe thickness threshold value).

In some embodiments, the safe threshold value is determined according to calibrated data, for example, a baseline value. Optionally, the calibration data is obtained from the patient herself, by measuring the thickness of the uterine wall (at one or more locations) away from the septum. Alternatively or additionally, the calibration data is obtained from measurements on a group of patients. Alternatively or additionally, the safe thickness threshold has been deduced from observational data, for example, according to a risk of uterine rupture.

In some embodiments, the measured thickness of the uterine wall is compared to the baseline thickness. Optionally, output is provided if the measured thickness is higher or lower or about the same as the baseline thickness. Potentially, the septum can be removed with the resulting wall thickness being about the same as the thickness of the rest of the nearby uterine wall.

In an exemplary embodiment of the invention, the uterus is filled with fluid (e.g., saline). Optionally, the filling of fluid distends the uterus.

In an exemplary embodiment of the invention, the measurement of the thickness of the ultrasound wall is performed with the ultrasound transducer positioned away from the uterine wall, without contacting the uterine wall (e.g., directly contacting the wall, or contacting coupling gel in contact with the wall). In an exemplary embodiment, the US imaging is performed through the fluid.

In some embodiments, the method comprises providing visual output of the area being surgically treated and/or the area being measured (e.g., overlapping areas). Alternatively, no visual output is provided, for example, the procedure is performed ‘blindly’.

An aspect of some embodiments of the invention relates to a method of aligning the tissue thickness measurement device. In an exemplary embodiment of the invention, the method comprises aligning a visual field and an ultrasound field so that measurements of tissue thickness are performed inside the visual field.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

### Overview

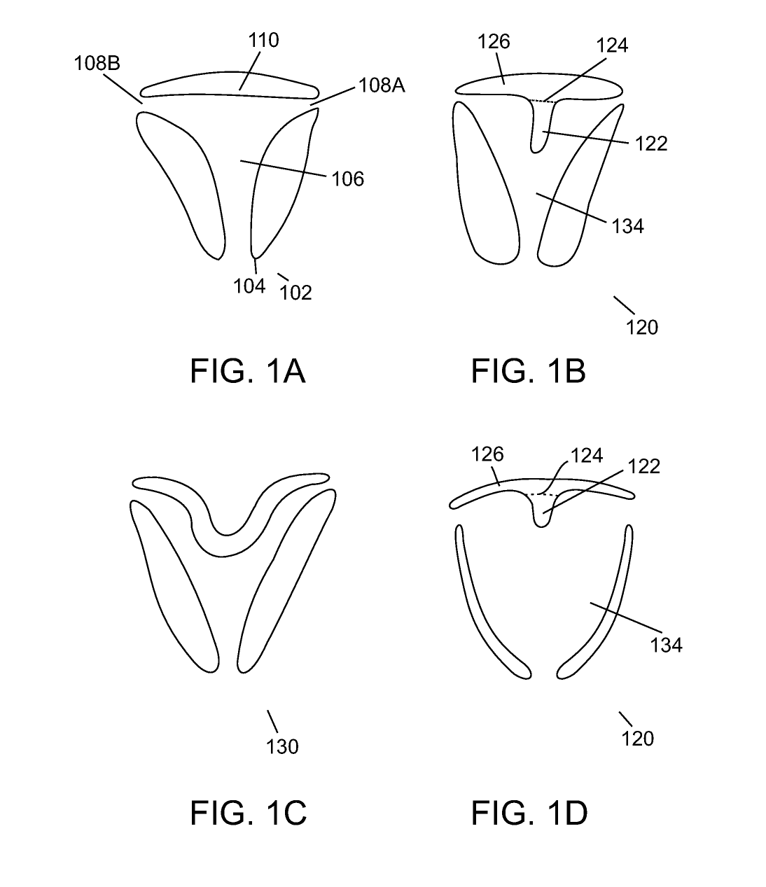

FIGS. 1A-1D are simplified schematics of anatomical variations of the uterus, useful to help understand an exemplary embodiment of the invention. The uterus is shown in a coronal cross section (e.g., as viewed facing the front of the body). For simplicity, surrounding structures have been omitted, for example, fallopian tubes, the bladder, intestines, ligaments and other external organs.

FIG. 1A is a simplified schematic of a uterus 102 that developed normally (e.g., without congenital abnormality or deformations). Uterus 102 comprises of a cervix 104 (shown dilated), potentially useful as an access port for insertion of the probe into a uterine cavity 106, in accordance with an exemplary embodiment of the invention. Uterus 102 also comprises a smooth fundus 110 (e.g., back wall). Fallopian tubes attached to uterus 102 at locations 108A-B have been removed for clarity.

FIG. 1B is a simplified schematic of a uterus 120 with a septum 122 extending from a fundus 126 (e.g., back wall), a common congenital malformation. In some cases, septum 122 is believed to have one or more effects, for example; infertility, early pregnancy loss, late abortion, premature delivery. In some cases, surgical removal of septum 122 (boundary with the fundal wall schematically illustrated by line 124) improves pregnancy rates. However, removal of septum 122 can be associated with risks, for example; perforation of the uterine wall (which can lead to intra-abdominal hemorrhage and/or bowel injury) and/or inadequate removal of the septum (which can require another procedure).

FIG. 1C is a simplified schematic of a bicornuate uterus 130. The uterine cavity of bicornuate uterus 130 can look similar to the uterine cavity of septate uterus 120. Under some imaging modalities (e.g., hysterosalpingography) and/or incorrect imaging (e.g., using ultrasound), it can be difficult to distinguish between the two types of malformations, and/or an incorrect diagnosis can be made. The bicornuate utertus 130 should not be treated to remove an incorrectly diagnosed septum.

FIG. 1D is a simplified schematic of septate uterus 120, after uterine cavity 134 has been distended, for example, filled with a fluid. Distension can change the geometry of fundal wall 126 and/or septum 122. For example, measurements obtained before the distension (e.g., using ultrasound) may not reflect changes in the dimensions due to the distension. For example, determining where septum 122 ends at boundary 124 can be difficult, which can increase the risk of perforation of the uterine wall, or alternatively, the septum may not be sufficiently removed.

Furthermore, in some women, the uterus varies from the most common anatomical position. For example, the uterus can be in various degrees of retroflexion or anteflexion. During distension, the geometry of the uterus can significantly change, affecting the measurements.

It should be noted, that the device and/or methods according to some embodiments of the invention can also be used to treat intra-uterine conditions other than the septum. For example, resection of fibroids (e.g., benign tumors originating from the uterine wall that protrude inside the uterine cavity. For example, submucous type fibroids, having part of the fibroid inside the uterine wall. For example, adhesions (e.g., result of traumatic curettage), even if the anatomy inside the uterus is difficult.

### Exemplary System

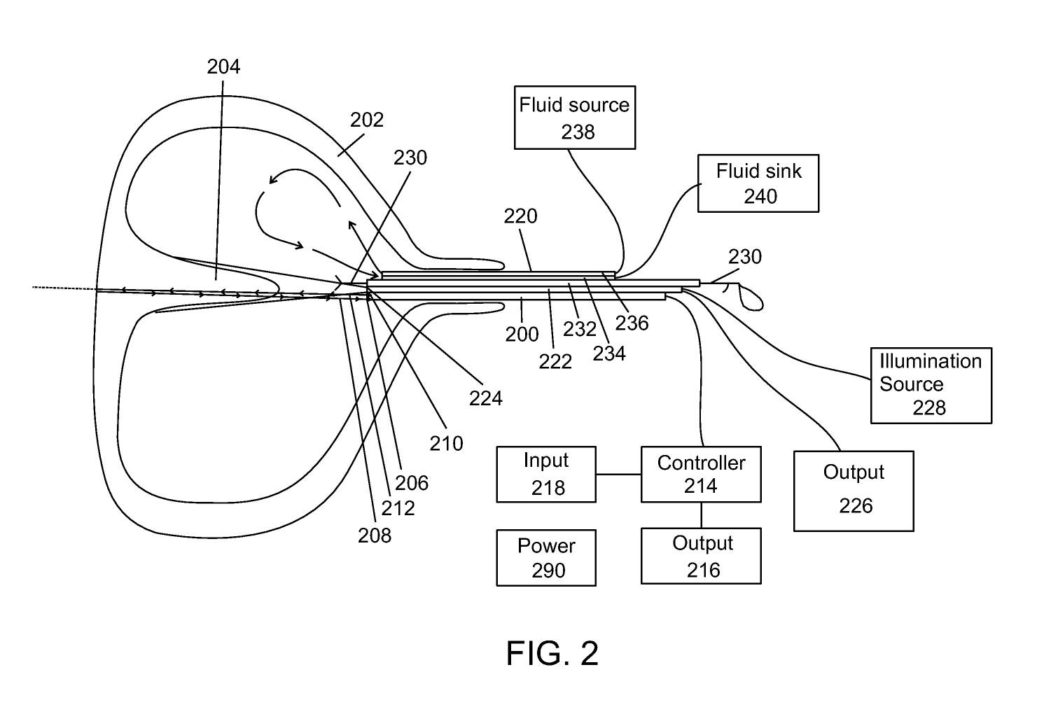

FIG. 2 is a schematic representation of a uterine probe 200, in accordance with an exemplary embodiment of the invention. Probe 200 is shown inserted in uterus 202, for example to help with surgical treatment of a uterine abnormality, for example, septum 204.

In an exemplary embodiment of the invention, probe 200 comprises at least one ultrasound receiver 206 adapted to receive ultrasound energy 208, for example, ultrasound reflected from tissues. Optionally or additionally, probe 200 comprises at least one ultrasound emitter 210 adapted to emit ultrasound energy 212.

In an exemplary embodiment of the invention, US receiver 206 is in electrical communication with a controller 214. Not necessarily limiting examples of electrical coupling include; a direct wired connection, a wireless connection, a network connection (e.g., internet, cellular network). The connection can be permanent (e.g., hard wired) or temporary (e.g., plug in).

In an exemplary embodiment of the invention, controller 214 is adapted to analyze signals from US receiver 206 to determine the thickness of the wall of uterus 202. Optionally, controller 214 estimates the boundary of fluid in the uterus and the inner uterine wall (e.g., first reflected signal). Optionally or additionally, controller 214 estimates the boundary of the outer uterine wall and tissues external to the uterus (e.g., second reflected signal). In an exemplary embodiment of the invention, controller 214 estimates the thickness of the uterine wall by the distances between the first and second reflected signals (e.g., converting time between the signals to distance according to the estimated speed of sound through the tissues and/or fluid).

In an exemplary embodiment of the invention, controller 214 is adapted to control ultrasound emitter 210, in order to control the output of ultrasound energy. For example, controller 214 controls the frequency and/or intensity of the emitted ultrasound energy.

In an exemplary embodiment of the invention, controller 214 comprises circuitry, for example, a circuit board embedded in probe 200 (e.g., application specific integrated circuit, ASIC). Alternatively or additionally, controller 214 is an external box, for example, a control station that plugs into probe 200. Alternatively or additionally, controller 214 comprises software, for example, residing on a laptop computer, a smartphone, or a remote server.

In some embodiments, controller 214 comprises a built in memory (e.g., random access memory, read only memory) to store gathered data, for example, US data, thickness threshold values.

In an exemplary embodiment of the invention, controller 214 is electrically coupled to one or more output elements 216. Optionally, output element 216 is adapted to provide visual and/or auditory output, for example, of the wall thickness. Not necessarily limiting examples of output elements 216 include; a display of a number indicating the thickness of the wall (e.g., in millimeters), a verbal message stating the thickness of the wall, lights (e.g., colored, flashing) corresponding to the thickness of the wall.

Alternatively, in some embodiments, controller 214 does not estimate the wall thickness directly. Optionally, controller 214 processes the signals from ultrasound receiver 206 to produce an image and/or sounds on output 216. Optionally, the measurement of the wall thickness is performed manually, for example, by the physician measuring the wall thickness on an image on a screen, or by the physician listening to beeps warning about the wall thickness.

In some embodiments, an input 218 is in electrical communication with controller 214. For example; a keyboard, a mouse, a touch screen, a laptop, a smartphone. Optionally, input 218 allows the user to program controller 214, for example, to set one or more wall thickness thresholds and/or select modes of output. Alternatively, the settings are preselected, for example, by the manufacturer.

In an exemplary embodiment of the invention, probe 200 is coupled to a sheath 220 (e.g., hysteroscope) inserted into uterus 202. For example, probe 200 is inserted in a lumen of sheath 220 or probe 200 is externally attached to sheath 220. In some embodiments, sheath 220 is hard and/or rigid. In other embodiments, sheath 220 is soft and/or flexible, for example, allowing improved maneuverability.

In an exemplary embodiment of the invention, probe 200 is aligned with a visual element 224 (e.g., disposed at a distal end of a visual probe 222, or probe 222 is integrated with sheath 220). Optionally, probe 222 comprises a source of illumination 228. In one example, probe 222 is a fiber optic visualization device. Optionally, visual probe 222 is in communication with an output interface 226 adapted to display visual output from visual element 224, for example, a monitor.

In an exemplary embodiment of the invention, probe 200 and probe 222 are aligned so that the field of view as seen from visual probe 222 encompasses the entire area imaged by probe 200 (when inside the uterus). For example, the area imaged by ultrasound probe 200 is about 10% of the visual area, or about 30%, or about 50%, or about 70%, or about 90%, or other smaller, intermediate or larger values. Optionally, probes 200/222 are aligned so that the area imaged by the ultrasound is approximately centered within the visual field of view. However, some offsetting is allowed, and may occur in practice as the distance to the wall is adjusted.

In an exemplary embodiment of the invention, probe 200 and probe 222 are coupled to each other so that movement of one probe (e.g., towards or away from the tissue, angular motion relative to the tissue) retains the alignment between the probes within an allowable tolerance. For example, probes 200 and 222 are mechanically attached to one another, for example, using clips. Alternatively, probes 200 and 222 are inserted into lumens of sheath 220, which retains the alignment.

In an exemplary embodiment of the invention, probe 200 is aligned with a cutting tool 230, so that tool 230 cuts within the area imaged by the US energy of probe 200. Optionally or additionally, tool 230 is aligned with the visual field of probe 222, so that tool 230 cuts within the visual field. For example, portion of tool 230 that cuts is positioned approximately in the center of the visual field. Not necessarily limiting examples of cutting tools 230 include; microscissors, lasers, electrosurgical devices (e.g., loops, either monopolar or bipolar). Optionally, tool 230 is inserted through a lumen 232 in sheath 220, the alignment is optionally performed by the lumens.

In some embodiments, lumens are provided to help with distension of the uterus. Optionally, sheath 220 comprises a fluid insertion lumen 236. Optionally, inflation lumen 236 is in fluid communication with a fluid source 238 adapted to provide a controlled flow of fluid. Optionally or additionally, sheath 220 comprises a fluid removal lumen 234. Optionally, fluid removal lumen 234 is in fluid communication with a fluid sink 240 adapted to remove a controlled flow of fluid.

In an exemplary embodiment of the invention, a power source 290 provides electrical energy to one or more elements of the system; electrical energy to power ultrasound emitter 210, ultrasound receiver 206, controller 214, output 216, output 226, input 218, illumination source 228, pumps and/or valves associated with fluid source 238 and/or fluid sink 240.

### Exemplary Ultrasound Parameters

Additional details of the ultrasound emitter and/or receiver are provided, for example, with reference to FIG. 2.

In an exemplary embodiment of the invention, the ultrasound emitter emits ultrasound with an energy intensity not sufficient for imaging. Optionally, the energy intensity is fairly low, for example, to prevent tissue damage from excessive heating of the tissue from the emitted energy.

In some embodiments, a single ultrasound element serves as the emitter and transmitter. Optionally, the US element emits the US in pulses separated by a delay, with the receiver sensing the feedback between the pulses.

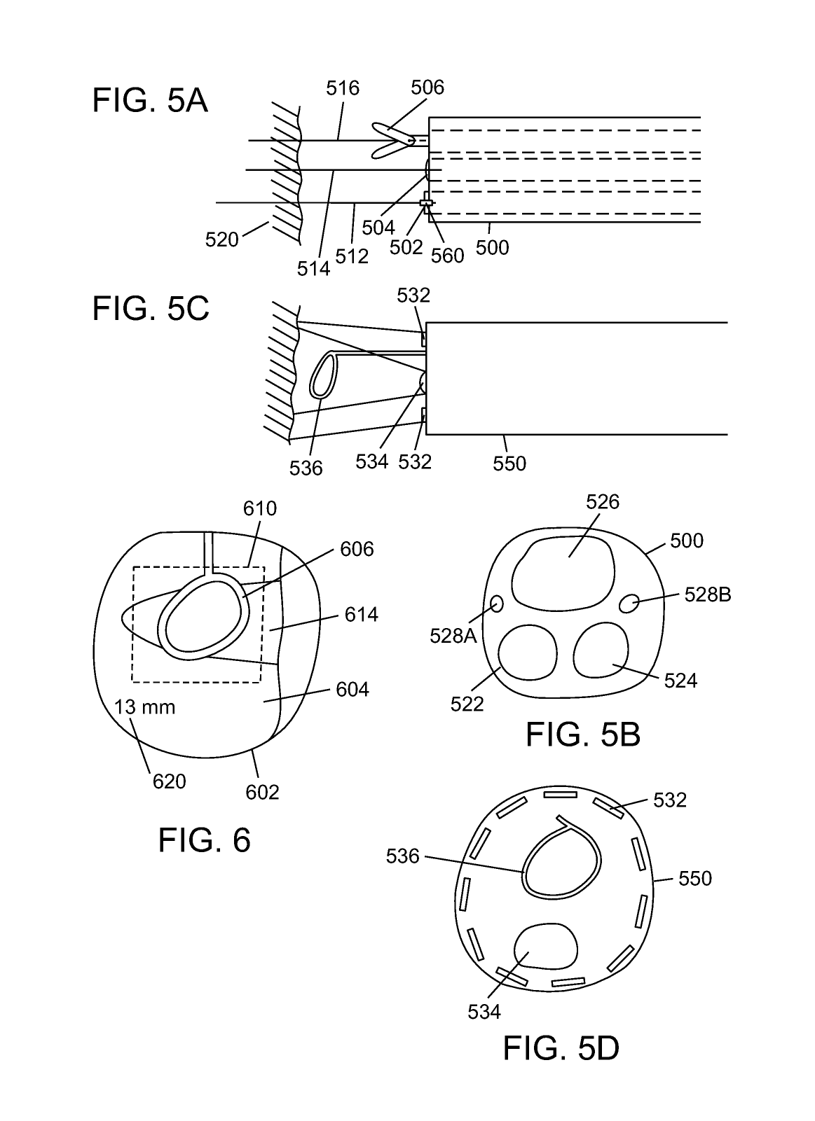

In some embodiments, a plurality of US elements are used. For example, 2, 4, 6, 8, 10, 20, 50, 100, 1000, or other smaller, intermediate or larger numbers of ultrasound elements. The US elements can be arranged in various configurations, not necessarily limiting examples include; an annular array, a linear array. Optionally, the US elements are arranged as a phased array. Potentially, the US arrangements help is aligning the US field with the visual and/or cutting fields, for example, as will be described with reference to FIGS. 5A-D.

In an exemplary embodiment of the invention, the total area of the US elements is about 1 mm², about 2 mm², about 4 mm², about 6 mm², about 10 mm², about 15 mm², about 20 mm², or other smaller, intermediate or larger areas. Optionally, the total area is small enough to fit into the uterus through the cervix, for example, as part of a hysteroscope.

In an exemplary embodiment of the invention, at least some of US elements are approximately round (e.g., when viewed face on). Alternatively or additionally, at least some of the US elements are square and/or rectangular. Alternatively or additionally, other shapes can be used. In some embodiments, the shape of the US elements is selected to match the shape of the visual field to help with alignment.

In some embodiments, the controller performs other functions, for example, steering of the US beam, selection of some US elements out of the total amount. Potentially, steering the field helps with alignment.

In an exemplary embodiment of the invention, the US elements are made out of a suitable material, for example; piezoelectric materials such as ceramic PZT.

In some embodiments, a matching layer is disposed on the exposed surface of one or more of the US elements, potentially, to help with energy transfer from the US element to the fluid in the uterus.

In some embodiments, a damping layer is disposed on the back surface of one or more of the US element, potentially, to help with reducing ringing of the US element.

In some embodiments, the US beam is at least somewhat focused on to the uterine wall, for example, the focal area encompasses the thickness of the uterine wall. Optionally, the focal length is adjustable. Not necessarily limiting examples of focusing include; an acoustic lens, a concave shape of the US element and/or a concave arrangement of a plurality of US elements, electronic focusing using the phased array. Alternatively, no special focusing is used. Potentially, adjusting the focal length helps with alignment of the US and visual fields.

### Exemplary Method of Operation

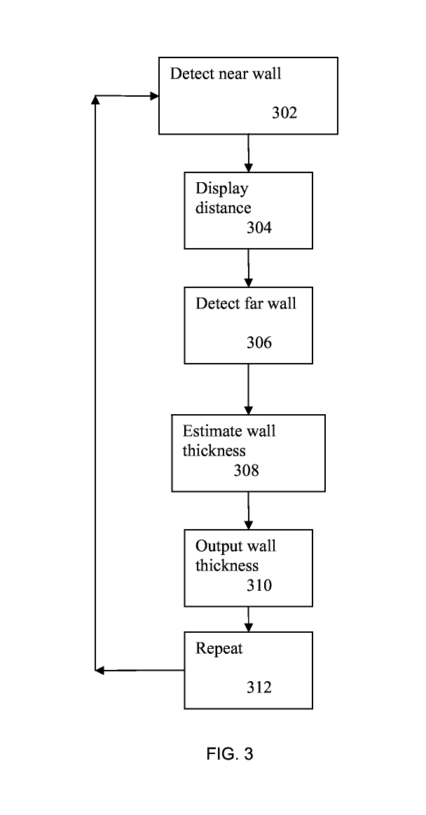

FIG. 3 is an exemplary method of estimating the thickness of the uterine wall, in accordance with an exemplary embodiment of the invention. The method can be used with the uterine probe, for example, as described with reference to FIG. 2. Optionally, the functions are performed by the controller. Alternatively, other devices can also be used. Furthermore, the method is exemplary, as some steps are optional.

At 302, the near wall of the uterus is detected. Optionally, the near wall is detected by sensing one or more echoes of emitted ultrasound energy. Optionally, the near wall of the uterus is the surface of the endometrium. Alternatively, the US settings are not good enough to properly distinguish the endometrium, in which case the near wall is the surface of the myometrium.

Optionally, the near wall is detected without with US emitter being in contact with the near wall. For example, fluid in the uterus bridges the acoustic gap between the US emitter and the near wall. Alternatively, the US emitted contacts the near wall.

Optionally, at 304, the distance to the near wall is estimated. Optionally, the distance is estimated by correlating the time of flight of the echo (e.g., from transmission to receiving) with the speed of sound in fluid.

Optionally, the distance to the near wall is outputted. For example, visually displayed on a screen (e.g., the number is displayed), a prerecorded number is played, lights flash at a rate corresponding to the distance.

Potentially, providing the distance to the near wall to the physician helps the physician get accustomed to the visual display of the system. Potentially, providing the distance to the near wall helps the physician determine how close to position the hysteroscope before pushing the cutting tool forward to contact the tissue.

At 306, the far wall of the uterus is detected. Optionally, the far wall is detected by sensing one or more echoes of the emitted ultrasound energy. Optionally, the far wall corresponds to the outer border of the myometrium.

Optionally, the far wall is detected without with US emitter being in contact with the near and/or far wall. For example, fluid bridges the acoustic gap between the US emitter and the near and/or far wall. Alternatively, the US emitted contacts the near and/or far wall.

Optionally, the far wall is detected, even if the intermediate tissue between the near and far wall is not homogenous. For example, tissue of the septum may comprise of endometrium, connective tissue and/or other tissue types.

At 308, the thickness of the uterine wall is estimated. Optionally, the thickness includes the thickness of the septum together with the thickness of the uterine wall.

Optionally, the thickness is estimated, for example, by determining the time from the first echo (of the inner wall) to the second echo (of the outer wall), and converting the time to distance according to the estimated speed of sound in tissue and/or fluid. Another possible method for estimating the thickness is using frequency modulated ultrasound, for example, correlating the frequency of the US echo with distance. Other suitable US based methods can also be used.

Alternatively, in some embodiments, the thickness is the thickness of the uterine wall alone, for example, if establishing a baseline for subsequent measurement comparisons. In some embodiments, the baseline is first established by the user pointing the probe towards the uterine wall and away from the septum, then pressing a button to record the natural uterine wall thickness in a memory of the controller.

In some embodiments, the thickness within one region (e.g., within the visual field) is measured. Alternatively, in some embodiments, the thickness within a plurality of regions is measured. Optionally, the thickness comprises the smallest value within the regions. Alternatively or additionally, the thickness comprises the average value within the regions.

In some embodiments, one or more slopes are calculated, between one or more sensed regions. For example, a positive slope can indicate the edge of the target tissue. For example, an approximately zero slope can indicate the uterine wall (or after removal of tissue). For example, a negative slope can indicate too much cutting of the target tissue and/or cutting into the wall, potentially a risk of perforation.

Optionally, at 310, the thickness of the uterine wall is outputted.

Optionally, the thickness of the uterine wall is compared to one or more predetermined threshold values. In one example, the threshold value is the lowest wall thickness, past which the surgeon should not continue cutting (e.g., to prevent or reduce the risk of perforation). For example, about 3.5 mm, about 5 mm, about 7 mm, about 10 mm, about 12 mm, or other smaller, intermediate or larger values. The threshold value is the baseline measured value, selected by the physician, or pre-programmed by the manufacturer. Optionally, the output relates the actual thickness to the threshold value, for example, output above the threshold (e.g., green light, verbal OK to continue, spaced apart beep sounds) and a different output below the threshold (e.g., red flashing light, verbal STOP, loud continuous beep). In another example, several ranges are used, for example, a first range that is associated with continued cutting (e.g., green light, verbal OK), a second range is associated with a warning to cut carefully (e.g., yellow light, verbal WARNING), and a third range that is associated with a signal to stop cutting (e.g., red light, verbal STOP). Alternatively, in another example, the output changes continuously according to the thickness. For example, as the wall is cut and the thickness is reduced, lights can begin to flash faster, beeps can be closer together and louder, the measured distance is displayed to the user.

Optionally, at 312, one or more boxes (e.g., 302, 304, 306, 308, 310) are repeated. Optionally, the boxes are continuously repeated, for example, ultrasound is continuously emitted, sensed and analyzed, with the output continuously updated. Alternatively, the boxes are repeated periodically, for example, ultrasound is emitted in bursts, sensed and analyzed, with the output updated periodically. Alternatively, the ultrasound is emitted continuously, with sensing and analysis occurring with a predetermined sampling rate, with the output updated periodically. For example, the output of the wall thickness is updated 10 times per second, or 5 times per second, or every second, or every 2 seconds, or every 5 seconds, or other smaller, intermediate or larger time frames. Alternatively, the boxes are repeated only upon a change in position of the probe (e.g., change in position triggered by a position sensor) and/or a change in position of the cutting tool (e.g., trigged by a position sensor). For example, the wall thickness is recalculated only if the probe is moved to a new location, or only if the cutting tool is being manipulated during cutting.

### Exemplary Method of Treatment

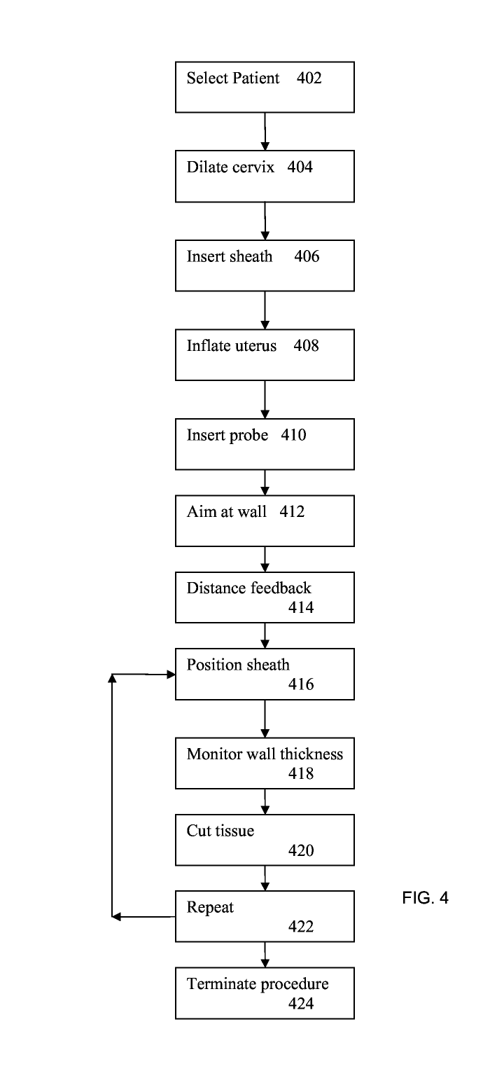

FIG. 4 is a flowchart of an exemplary method of surgically treating a patient with monitoring of the thickness of the uterine wall from inside the uterus, in accordance with an exemplary embodiment of the invention. The method is exemplary, as some boxes are optional, and/or some boxes can be performed in a different order.

Optionally, at 402, a patient with a uterine abnormality is selected for treatment, for example, by the physician.

Optionally, the patient is selected according to anatomical indications. For example, a septate uterus, a fibroid, adhesions, requiring operative hysteroscopy.

Optionally or additionally, the patient is selected according to clinical indications. For example, the patient suffered from one or more symptoms; recurrent pregnancy loss, infertility.

Optionally, at 404, the cervix of the patient is dilated, for example, using sequentially larger diameter dilators. The cervix is dilated to allow insertion of a hysteroscope that includes the ultrasound probe to measure the thickness of the uterine wall. The cervix is dilated to, for example, up to about 5 mm, or 8 mm, or 10 mm, or 12 mm, or 15 mm or other smaller, intermediate or larger diameters. Alternatively, no dilation is required. For example, the cervix opening is naturally large enough for the hysteroscope to fit and/or the hysteroscope is small enough.

Optionally, at 406, the sheath (e.g., hysteroscope) is inserted into the uterine cavity through the cervix. In some embodiments, the hysteroscope is inserted without the ultrasound probe (e.g., if the probe is a separate device). Alternatively, in some embodiments, the hysteroscope is inserted together with the ultrasound probe.

Optionally, at 408, the uterine cavity is inflated. Optionally, an inflation fluid is inserted through one or more lumens of the hysteroscope. Not necessarily limiting examples of fluids include; 1.5% glycine solution, isotonic saline. Optionally, the inflation fluid is selected by the physician, for example, according to clinical and/or surgical considerations. Optionally, the inflation fluid is circulated within the uterine cavity, and the fluid is removed through one or more lumens of the hystero scope.

At 410, the ultrasound probe is inserted into the uterine cavity. Optionally, the ultrasound probe is inserted into a lumen of the hysteroscope, the hysteroscope already having been inserted into the uterine cavity in 406. Alternatively, the US probe is inserted alone, e.g., without other guiding and/or visualization devices.

In an exemplary embodiment of the invention, the ultrasound probe does not need to contact the uterine tissue in order to image the tissue. Optionally, imaging is performed through the fluid in the uterine cavity, for example, the fluid providing an acoustic window. The ultrasound probe is maintained at a distance away from the uterine wall (e.g., during imaging of the wall) of about 3 mm, or about 5 mm, or about 7 mm, or about 10 mm, or about 15 mm, or other smaller, intermediate or larger distances.

Optionally, at 412, the hysteroscope is aimed at the target tissue to be cut and/or removed. For example, the hysteroscope is positioned towards the uterine septum. Optionally, the percentage of the septum occupying the uterine cavity is estimated, for example, from about 20%-100% (e.g., total uterine septum). Optionally, the aiming is performed visually, for example, by using the fiber optic viewer to examine the interior of the uterine cavity. Alternatively, the aiming is performed without visual feedback.

Optionally, at 414, the distance from the hysteroscope to the target tissue is monitored, for example, by the physician obtaining feedback associated with the distance from the output element.

Optionally, at 416, the hysteroscope is positioned relative to the target tissue in close enough proximity to cut the tissue. For example, an electrical cutting loop is positioned against the septum. Optionally, the positioning is based according to the distance measured at 414, for example, the physician adjusts the position of the hysteroscope until the hysteroscope is close enough to the tissue.

At 418, the thickness of the uterine wall aimed at the by the hysteroscope is monitored. For example, the physician is provided with feedback associated with the thickness of the uterine wall, for example, as described with reference to FIG. 3.

In some embodiments, the thickness of the uterine wall is estimated before cutting begins. Potentially, measuring the thickness of the wall helps to make sure that the cutting tool is aimed at the abnormal tissue (e.g., septum) and not at the uterine wall.

Alternatively or additionally, in some embodiments, the thickness of the uterine wall that does not include abnormal tissue is measured. Optionally, the measured thickness is used as a threshold during cutting of the septum, for example, as described herein.

In some cases, the measured wall thickness varies according to the position of the hysteroscope relative to the tissue. For example, changing the angle of the hysteroscope relative to the target tissue (e.g., using the tip of the hysteroscope at the pivot point) may provide different measurements of the wall thickness. For example, lateral displacement of the hysteroscope along the tissue wall may provide different thickness measurements. For example, forward and/or reverse displacement of the hysteroscope relative to the wall will most likely not provide different thickness measurements.

At 420, a portion of the (e.g., abnormal) target tissue is resected. For example, using scissors, laser and/or loop electrosurgery. Optionally, the amount cut is sufficiently small so as not to perforate the uterine wall, for example, the amount cut is smaller than the most recent thickness measurement.

In some embodiments, at 422, one or more boxes are repeated. Optionally, 416 is repeated to reposition the hysteroscope relative to the target tissue. For example, if the wall thickness is too small but some septum remains, or if the septum needs to be cut at a different location and/or different angle. Optionally, 418 is repeated to re-measure the wall after the portion has been cut. Alternatively, in some embodiments, 418 and 420 are performed simultaneously, for example, to estimate the thickness of the wall as the septum is cut.

In other embodiments, instead of repeating (e.g., 422), the procedure is terminated. Optionally, the procedure is terminated once the septum (or other tissues) has been sufficiently cut (e.g., until the myometrium), for example, to the satisfaction of the physician. Alternatively, the procedure is terminated if no more cutting can be performed safety, for example, the safety threshold thickness has been reached.

Optionally, at the termination of the procedure, the hysteroscope is removed from the uterus. Optionally, the fluid in the uterus is allowed to drain out. Optionally, the ultrasound probe is removed from the hysteroscope and disposed.

In some embodiments, the ultrasound probe (e.g., 410) is inserted into the uterine cavity together with the hysteroscope as in 406. Optionally, the distance to the wall is monitored (e.g., 414) as the probe is being inserted (e.g., 406), for example, to prevent contact with the uterine wall.

### Some Potential Advantages of Some Embodiments

The following are one or more potential advantages of some embodiments:
Reduction or prevention of perforation of the uterine wall, for example, due to the measurements of the thickness of the uterine wall during cutting.
Better removal of excess tissues (e.g., septum, fibroids), reducing or preventing the need for reoperation and/or two step surgeries.
Improved pregnancy rates due to the improved removal of the uterine septum.
Improved measurement of uterine wall thickness while the uterus is distended and during the procedure.
Reduction in operation time, for example, as measurement of the uterine wall is performed automatically and/or continuously during the procedure, so that the physician does not need to cut, stop and measure, and then cut again.
Laparoscopy to assist with the procedure, for example, to examine the uterine fundus for perforations, may not be required.
Reduction in risk of uterine rupture in subsequent pregnancies, for example, by using the device to prevent or reduce excessive cutting of the fundal wall.
Reduction in risk of perforation and/or improved outcomes in difficult anatomical situations, for example, a fibroid partially in the uterine wall, uterine adhesions preventing full distension of the uterine cavity.
Assistance with performance of ‘blind’ procedures, for example, curettage. Potentially, improving outcomes and/or reducing the risk of perforation.

### Exemplary Distal Tip of Sheath

FIGS. 5A-5D are simplified schematics of some distal tips of the sheath for insertion in the uterus, for example, the distal tips of a hysteroscope. In an exemplary embodiment of the invention, the distal tip is arranged to provide alignment of the cutting tool, the visual field and/or the area of the uterine wall being measured. Optionally, the alignment helps to ensure that the thickness of the wall being monitored corresponds to the tissue being cut. Optionally or additionally, the alignment helps to ensure that the area being cut is visualized.

FIG. 5A is a schematic of a side view of a distal tip of a sheath 500 to illustrate parallel alignment, in accordance with some embodiments of the invention. The distal tip is shown placed in close proximity to target tissue 520. In an exemplary embodiment of the invention, the distal tip comprises at least one ultrasound element 502 (e.g., emitter and/or receiver). Optionally or additionally, the distal tip further comprises at least one visual element 504 (e.g., fiber optic bundle). Optionally or additionally, the distal tip further comprises at least one cutting tool 506. Dotted lines through sheath 500 illustrate a plurality of lumens and/or probes in the lumens and/or built in devices for ultrasound element 502, visual element 504 and/or tool 506.

In some embodiments, an ultrasound axis 512 (e.g., imaginary line through the middle of the ultrasound beam, for example, perpendicular to the ultrasound element) is in parallel with a visual axis 514 (e.g., imaging line through the middle of the visual field). Optionally or additionally, a cutting axis 516 (e.g. imaginary line showing where tissue will be cut) is in parallel with ultrasound axis 512 and/or visual axis 514.

In some embodiments, the distance between any two axes is small enough that monitoring the thickness of tissue and/or visually viewing the tissue next to the tissue being cut is sufficient to prevent perforation by the cutting. The distance between any two axes is no more than, for example, about 0.1 mm, or about 0.3 mm, or about 0.5 mm, or about 1 mm, or about 2 mm, or about 3 mm, or other smaller, intermediate or larger distances.

In some embodiments, a light element 560 is positioned so that the emitted light indicates the US field. Optionally, element 560 is colored, for example, blue, green or other colors. Optionally, element 560 is coaxially positioned with US element 502 (e.g., in a hole in the middle of US element 502). Optionally, the light field produced by light element 560 is approximately aligned with the US field produced by US emitted 502. Potentially, viewing the colored light (e.g., using visual element 504) provides an indication of the location of the US field and of the tissue being measured.

In some embodiments, US element 502 is axially displaceable relative to sheath 500, for example, by pulling or pushing US element 502 in a lumen of sheath 500. Optionally, the axial displacement is finely controlled, for example, manually (e.g., by rotating a screw) and/or automatically by software. Optionally, the axial movement is used to control the spread of the US beam, for example, displacing US element 502 into sheath 500 reduces the spread of the beam, and moving US element 502 out of sheath 500 increases the beam spread. Potentially, increasing and decreasing the beam spread helps to align the US beam with the visual field and/or cutting fields. Alternatively

FIG. 5B is a face-on view of the distal tip, showing one possible arrangement for the lumens and/or probes. Optionally, cutting lumen 526 is the largest lumen, occupying a position towards the upper side of sheath 500. Optionally, US probe lumen 522 is smaller than cutting lumen 526, occupying a position below cutting lumen 526 and towards one side. Optionally, visual probe lumen 524 is smaller than cutting lumen 526, and either smaller, larger or about the same size as US probe lumen 522. Optionally, in some embodiments, irrigation lumens 528A-B are used to insert and remove fluid from the uterine cavity.

Alternatively, in some embodiments, the lumens are of different sizes and/or proportions. Alternatively, in some embodiments, the lumens are lined up along an axis on the face of the distal tip.

Potentially, the parallel embodiments provides for a simpler and/or cost effective design while still adequately monitoring the thickness of the wall during cutting.

FIG. 5C is a simplified schematic of a side view of a sheath 550, to help illustrate overlapping alignment, in accordance with some embodiments of the invention.

In some embodiments, sheath 550 comprises one or more ultrasound elements 532. Optionally, the ultrasound beam generated by elements 532 encompasses the entire area that can be accessed by tool 536, for example, the entire surface of tissue being contacted and/or treated by tool 536.

In some embodiments, sheath 550 comprises one or more visual elements 534. Optionally, the field of view from visual elements 534 encompasses the entire area that can be accessed by tool 536. Optionally or additionally, the field of view overlaps with the area being sensed by ultrasound elements 532. Optionally, the field of view is larger than the area being sensed. Alternatively, the field of view is smaller than the area being sensed.

Potentially, the overlapping embodiments provide increased assurance that the tissue being cut is being entirely viewed and/or being monitored for wall thickness.

FIG. 5D is a simplified schematic of a face on view of sheath 550.

In some embodiments, ultrasound elements 532 are integrated with sheath 550. Optionally, ultrasound elements 532 are disposed along an outer perimeter of the distal end of sheath 550. Optionally, elements 532 are a plurality of small US elements arranged long the outer circumference. Alternatively, one ring shaped US element is used.

In some embodiments, visual element 534 is integrated with sheath 550. In some embodiments, surgical tool 536 is integrated with sheath 550. Optionally, visual element 534 and/or surgical tool 536 are at least partially surrounded by US elements 532.

### Exemplary View

FIG. 6 is a simplified schematic of an image 602 as seen using the visual element of the sheath (e.g., fiber optic bundle) when inserted into the uterus to cut tissues (e.g., septum 604), in accordance with an exemplary embodiment of the invention. Optionally, image 602 is viewed on a monitor.

In an exemplary embodiment of the invention, the cutting portion of a cutting tool 606 (e.g., loop) is visible on image 602.

In an exemplary embodiment of the invention, the cutting is visible on image 602, for example, cut tissue 614 (shown as shaded) of septum 604.

In some embodiments, the area being monitored for the uterine wall thickness is displayed on the screen, for example, dashed line box 610. Potentially, displaying the area being monitored helps provide additional confidence to the surgeon that the wall will not be perforated by the cutting. Alternatively, the area being monitored is not displayed on the screen.

In some embodiments, the thickness of the uterine wall (e.g., within box 610) is displayed of image 604. For example, a numerical value 620 (e.g., in millimeters) of the thickness is shown. Optionally, value 620 is displayed with different colors, for example, corresponding to safety thresholds (e.g., green=safe to cut, yellow=cut carefully, red=wall too thin to continue cutting). Optionally, value 620 flashes, for example, if cutting is unsafe.

### Exemplary Method of Alignment

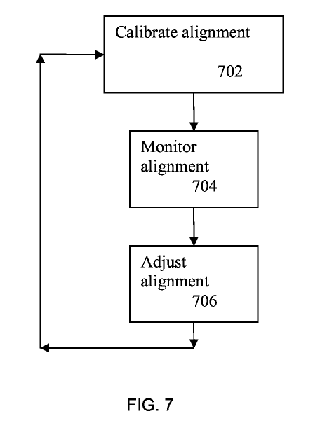

FIG. 7 is a flowchart of an exemplary method of aligning a tissue thickness measuring field (e.g., using an ultrasound filed) with a visual field. Optionally, the method also comprises aligning a cutting field. Optionally, the alignment method is used with other methods described herein (e.g., FIGS. 3 and/or 4) and/or other with device embodiments described herein.

At 702, the system is calibrated. Optionally, calibration comprises aligning the US field to measure tissue within the visual field. Optionally, calibration further comprises positioning the cutting instrument within the visual field and/or measuring field.

In some embodiments, the US field is adjusted, for example, by changing the focal length of a lens, blocking some of the spread of the US (e.g., as described with reference to FIGS. 5A-B) and/or using a phased array and/or an ultrasound element adapted to be stimulated in more than one way (e.g., stimulate the inside but not the outside).

In some embodiments, the visual field is adjusted, for example, by changing the focal length of a lens, and/or blocking the outer visual field (e.g., as described with reference to FIGS. 5A-B), for example, by moving the visual element in or out of a lumen in a hysteroscope.

In some embodiments, the cutting field is adjusted, for example, by selecting a suitably shaped and/or sized cutting tool and/or using cutting tool with a pivotal tip.

Optionally, calibration is performed automatically, for example, the user looks through the visual element and positions the target tissue within the visual field, then presses a button to calibrate the system. Alternatively, calibration is performed manually, for example, by the user looking through the device at the target tissue and manually adjusting the system, for example, lining up the colored light in the visual field (e.g., as described with reference to FIGS. 5A-B).

Optionally, at 704, the alignment is monitored. Optionally, the monitoring occurs during the cutting procedure.

In some embodiments, the monitoring is performed, for example, by automatically and/or manually looking for sudden changes in thickness and/or slope. Optionally, the changes are greater than would occur as a result of cutting. Alternatively, the changes can also be performed by cutting, but flagged anyways as a safety measure. Potentially, a sudden decrease or increase in thickness and/or slope suggests a misalignment (e.g., US field no longer pointed at target tissue). For example, monitoring is performed by looking for changes in the color of the visual field (e.g., manually by the user, automatically using image processing software).

Optionally, at 706, the system is re-aligned, for example, if an alignment problem is detected at 704. Optionally, re-aligning is performed using one or more methods as described in 702.

Optionally, the monitoring and aligning are performed repetitively and/or substantially simultaneously to maintain the alignment, potentially to help ensure that the measured thickness corresponds to the tissue being cut.

### Exemplary Kit

In an exemplary embodiment of the invention, probe 200 with ultrasound emitter 210 and/or ultrasound receiver 206 is sold separately from the rest of the hysteroscope. Optionally, probe 200 is sized to fit into a lumen of the hysteroscope. For example, having a diameter of about 1 mm, or about 2 mm, or about 3 mm, or about 5 mm, or other smaller, intermediate or larger diameters. Alternatively, probe 200 is adapted to attach to the external sheath of the hysteroscope, for example, using clip-on rings. Optionally, probe 200 is sold with controller 214 embedded therein. Optionally or additionally, probe 200 is sold with output 216 embedded therein. Optionally or additionally, probe 200 is sold with input 218 embedded therein.

In an exemplary embodiment of the invention, probe 200 is disposable, for example, for single use only. Optionally, probe 200 is sold pre-sterilized and packaged ready for use.

Alternatively, in some embodiments, the hysteroscope is sold, having probe 200 integrated therein. Optionally, the hysteroscope comprises controller 214 and/or output 216 capabilities integrated therein.

Alternatively, is some embodiments, software for controller 214 and/or output 216 is sold separately and/or is packaged with probe 200 and/or can be downloaded (e.g., from a website). Optionally, the software can be loaded onto a computer (e.g., laptop, smartphone), with probe 200 connected to the computer. Optionally, the software provides input 218 capabilities.

### Some Examples of Other Possible Applications

In some embodiments of the invention, the US probe as described herein is adapted for other locations in the body. For example, the US probe is not limited to be used only inside the uterus. In some embodiments, the US probe is adapted for measuring the thickness of other tissues, for examples, tissues that require careful cutting to prevent adverse outcomes (e.g., perforation).

In some embodiments, the US probe is adapted for use together with a urological device, for example, a cystoscope. In one example, the probe is adapted for imaging the thickness of the prostate, for example, with help during transurethral resection of the prostate (TURP). For example, the US elements are positioned on the exterior surface of the probe, pointing radially towards the cutting area of the prostate tissue. In another example, the probe is adapted to measure the thickness of the bladder wall, for example, for help during resection of bladder tumors.

In some embodiments, the region between the US element and the target tissue is filled with a fluid to help with imaging, for example, with saline.

In some embodiments, the US probe is adapted for use with a laparoscope. Optionally, the US probe is adapted to measure the thickness of the uterine wall from outside the uterus. For example, to help with procedures on the exterior of the uterus. Potentially, risk of perforation of the uterus (from the outside to the inside) is reduced and/or prevented.

### General

It is expected that during the life of a patent maturing from this application many relevant intrauterine probes will be developed and the scope of the term intrauterine probe is intended to include all such new technologies a priori. As used herein the term “about” refers to ±10%

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1. A probe sized for insertion into a uterus, said probe comprising:
at least one ultrasound element adapted to emit ultrasonic energy, said ultrasound element disposed on a distal end of said probe;
at least one ultrasound receiver adapted to receive ultrasound energy, said ultrasound receiver disposed on a distal end of said probe; and
circuitry adapted to automatically estimate a thickness of a uterine wall according to said received ultrasound energy,
wherein said at least one ultrasound element is arranged to be forward facing so that said ultrasound energy is directed to intersect a surgical site on said uterine wall.

2. The probe of claim 1, wherein said circuitry is non-imaging.

3. The probe of claim 1, wherein said circuitry is further configured to only measure said thickness.

4. The probe of claim 1, wherein said probe is sized for insertion into a lumen of a hysteroscope.

5. The probe of claim 1, wherein said uterine wall thickness comprises a thickness of a uterine septum and a myometrium.

6. (canceled)

7. The probe of claim 1, wherein said ultrasound element is arranged so that said ultrasound energy is directed within a visual field on said uterine wall.

8. The probe of claim 1, wherein said ultrasound element is arranged to image an area no larger than about 100 mm².

9. The probe of claim 1, wherein said ultrasound element is arranged to produce an ultrasound beam of frequency from about 3-12 Mhz.

10. The probe of claim 1, wherein said probe is coupled to at least one of a visual element and a cutting element so that said probe moves together with said visual element and said cutting element to maintain an intersection of an ultrasound sensing area and at least one of a visual field and a surgical field, during movement of said probe.

11. The probe of claim 1, further comprising an output element in electrical communication with said circuitry, said output element adapted to provide at least one of visual and auditory output of said thickness.

12. The probe of claim 1, further comprising a colored light source positioned coaxially with said at least one ultrasound receiver so that a colored light field at least partially overlaps with a region of said wall being measured.

13. The probe of claim 1, wherein said probe is sized for insertion through an undilated cervix.

14. A device for insertion into a uterus, said device comprising:
a probe as in claim 1;
at least one visual element adapted to provide visual images of a portion of a uterine wall, said visual element positioned so that a visual field encompass an ultrasound imaging field of said uterine wall; and
at least one lumen sized for insertion of a surgical tool.

15. The device of claim 14, wherein said at least one lumen is positioned so that said surgical tool treats said uterine wall within said visual field and within said ultrasound imaging field.

16. The device of claim 14, wherein said device is sized for insertion into said uterus through a cervix.

17. The device of claim 14, wherein said device is flexible for improved maneuverability within said uterus.

18-37. (canceled)

38. A system comprising:
a probe sized for insertion into a uterus, said probe comprising:
at least one ultrasound element adapted to emit ultrasonic energy in a forward facing beam which diverges less than about 15 degrees, said ultrasound element disposed on a distal end of said device;
at least one ultrasound receiver adapted to receive ultrasound energy, said ultrasound receiver disposed on a distal end of said device; and
an optical element configured to image a visual field, wherein said ultrasonic beam intersects or is in close proximity with said visual field; and
circuitry adapted to generate an image from said received ultrasound energy.

39. A system according to claim 38, comprising a display which shows said visual field and an area monitored by said beam.

40. A system according to claim 38, comprising a display which shows said generated image.

41. A system according to claim 38, wherein the probe comprises a plurality of lumens, which align an area imaged by said ultrasonic beam with a cutting tool.

42. A system according to claim 38, wherein the probe comprises a plurality of ultrasonic elements.

43. (canceled)

44. A probe according to claim 1, wherein a single ultrasonic element serves as both said an ultrasonic element to emit and as an ultrasonic receiver.

45. A probe according to claim 1, wherein said circuitry is configured to estimate said thickness when said ultrasonic element is not in contact with said location.
